(19) United States
(12) Patent Application Publication
(10) Pub. No.: US 2009/0216046 A1
(43) Pub. Date: Aug. 27, 2009

(54) SELECTIVE ACYLATION OF 4-SUBSTITUTED-1,3-PHENYLENEDIAMINE

(75) Inventor: Ronald Stanley Michalak, Congers, NY (US)

Correspondence Address: FITZPATRICK CELLA (WYETH), 30 ROCKEFELLER PLAZA, NEW YORK, NY, 10112-3800, US

(73) Assignee: WYETH, MADISON, NJ (US)

(21) Appl. No.: 12/091,352

(22) PCT Filed: Oct. 12, 2006
(86) PCT No.: PCT/US2006/040055

§ 371 Date: Oct. 2, 2008

Related U.S. Application Data
(60) Provisional application No. 60/733,609, filed on Nov. 4, 2005.

(51) Int. Cl. C07C 231/10

(52) U.S. Cl. 564/218

(57) ABSTRACT

This invention is directed to a method of selectively acylating a compound of formula (II): (II), wherein: R¹ is NO₂, —N⁺R³₃, trihalomethyl, —CN, —SO₃H, —CO₂H, —CO₂ R³, —CHO and —COR₃, wherein R³ is C₁-C₆ alkyl, C₁-C₆ haloalkyl, C₃-C₁₂ cycloalkyl, C₆-C₁₂ aryl, C₂-C₉ heteroaryl, or C₁-C₉ heterocycloalkyl; comprising the step of reacting the compound of formula (II) with an acylating reagent to form a compound of formula (I): (I), wherein R² is selected from C₁-C₁₂ alkyl, C₁-C₁₂ haloalkyl, C₂-C₇ alkenyl, C₂-C₇ alkynyl, C₃-C₁₂ cycloalkyl, C₆-C₁₂ aryl, C₁-C₉ heterocycloalkyl, C₂-C₉ heteroaryl, C₁-C₁₂ alkoxy, C₁-C₁₂ haloalkoxy, C₃-C₁₂ cycloalkoxy, C₁-C₉ heterocycloalkoxy, C₆-C₁₂ aryloxy, and C₂-C₉ heteroaryloxy; or salts thereof.

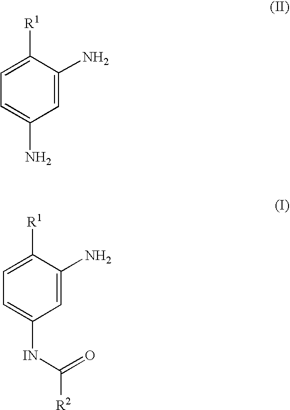

### BACKGROUND OF THE INVENTION

Field of the Invention

This invention is directed to a method of selectively acylating the 1-amino group of 4-substituted-1,3-phenylenediamine.

### DESCRIPTION OF RELATED ART

Related Background Art

Selective protection of functional groups can be a critical element in the synthesis of a complex molecule. For example, 4-Nitro-1,3-phenylenediamine is a useful inexpensive starting material for synthesizing larger molecules. However, a one-step route to 2-amino-4-acylated nitrobenzene requires a selective acylation of 4-nitro-1,3-phenylenediamine at the 1-amino position.

There are four isomeric nitrophenylenediamines with unsymmetrical amino substituents. A consideration of the relative electronic effects of induction and resonance successfully predicts one specific amino substituent in each of three of these isomers that is more nucleophilic in the presence of a variety of electrophiles. See U.S. Pat. No. 4,137,310; Shalaby et al., J. Org. Chem. 1996, 61, 9045-9048; Abasolo et al., J. Heterocyclic Chem. 1987, 24, 1771-1775; Harvey et al., J. Chem. Soc. Perkin Trans. I 1988, 694-696; and Rajuppa et al., Indian J. Chem. 1980, 19B, 533-535. These electronic arguments, however, are less clear in predicting the most nucleophilic amino substituent of 4-nitro-1,3-phenylenediamine. Acylation of 4-nitro-1,3-phenylenediamine using a mixture of acetic anhydride and acetic acid gave a 2:1 mixture of 2-amino-4-acetimidonitrobenzene and the diacetyl derivative. See Phillips, J. Chem. Soc. 1930, 1910-1916. Japanese Patent No. 09255636 discloses that 2-amino-4-acetimidonitrobenzene can be synthesized by selective cleavage of 1,3-bisacetamide-4-nitrobenzene. There has been no report of reaction conditions that selectively differentiate between the two amino substituents of 4-nitro-1,3-phenylene diamine.

The present invention provides the necessary reaction conditions to selectively acylate 4-substituted-1,3-phenylenediamine at the 1-amino position in high yield.

### BRIEF DESCRIPTION OF THE INVENTION

This invention is directed to a method of selectively acylating a compound of formula (II):

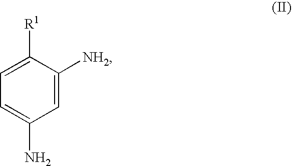

wherein:

R¹ is NO₂, —N⁺R³₃, trihalomethyl, —CN, —SO₃H, —CO₂H, —CO₂ R³, —CHO and —COR³, wherein R³ is C₁-C₆ alkyl, C₁-C₆ haloalkyl, C₃-C₁₂cycloalkyl, C₆-C₁₂ aryl, C₂-C₉ heteroaryl, or C₁-C₉ heterocycloalkyl;

comprising the step of reacting the compound of formula (II) with an acylating reagent to form a compound of formula (I):

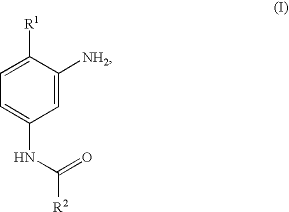

wherein R² is selected from C₁-C₁₂ alkyl, C₁-C₁₂ haloalkyl, C₂-C₇ alkenyl, C₂-C₇ alkynyl, C₃-C₁₂ cycloalkyl, C₆-C₁₂ aryl, C₁-C₉ heterocycloalkyl, C₂-C₉ heteroaryl, C₁-C₁₂ alkoxy, C₁-C₁₂ haloalkoxy, C₃-C₁₂ cycloalkoxy, C₁-C₉ heterocycloalkoxy, C₆-C₁₂ aryloxy, and C₂-C₉ heteroaryloxy;

or salts thereof.

### DETAILED DESCRIPTION

In the present invention compounds of formula (II) are selectively acylated at the 1-amino position to form compounds of formula (I). Using the conditions disclosed herein compounds of formula (I) can be produced in high yield in one step using acylating reagents such as, for example, acetyl chloride, acetic anhydride, ethyl chloroformate, benzoyl chloride and pivaloyl chloride.

The present method provides compounds of formula (I) in crude yields of at least about 60%. In one embodiment of the present method the compound of formula (I) is synthesized with a crude yield of at least about 70%. In another embodiment of the present method the compound of formula (I) is synthesized with a crude yield of at least about 80%. In the most preferred embodiment of the present method the compound of formula (I) is produced in a crude yield of about 90%.

For purposes of this invention the term “alkyl” includes straight chain moieties with a length of up to 12 carbon atoms, but preferably 1 to 8 carbon atoms, and more preferably 1 to 4 carbons. The term “alkyl” also includes branched moieties of 3 to 12 carbon atoms. The term “alkenyl” refers to a radical aliphatic hydrocarbon containing one double bond and includes both straight and branched alkenyl moieties of 2 to 7 carbon atoms. Such alkenyl moieties may exist in the E or Z configurations; the compounds of this invention include both configurations. The term “alkynyl” includes both straight chain and branched moieties containing 2 to 7 carbon atoms having at least one triple bond. The term “cycloalkyl” refers to alicyclic hydrocarbon groups having 3 to 12 carbon atoms and includes but is not limited to: cyclopropyl, cyclobutyl, cyclopentyl, cyclohexyl, cycloheptyl, norbornyl, or adamantly. Most preferred is where the cycloalkyl moiety contains 3 to 6 carbon atoms.

For purposes of this invention the term “aryl” is defined as an aromatic hydrocarbon moiety and may be substituted or unsubstituted. An aryl may be selected from but not limited to, the group: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl. An aryl may be optionally mono-, di-, tri- or tetra-substituted with substituents selected from, but not limited to, the group consisting of alkyl, acyl, alkoxycarbonyl, alkoxy, alkoxyalkyl, alkoxyalkoxy, cyano, halogen, hydroxy, nitro, haloalkyl, trifluoromethyl, trifluoromethoxy, trifluoropropyl, amino, alkylamino, dialkylamino, alkylaminoalkyl, dialkylaminoalkyl, hydroxyalkyl, alkylthio, —SO₃H, —SO2NH₂, —SO₂NHalkyl, —SO₂N(alkyl)₂, —CO₂H, CO₂NH₂, CO₂NHalkyl, and —CO₂N(alkyl)₂. Preferred substituents for aryl include: alkyl, halogen, amino, alkylamino, dialkylamino, trifluoromethyl, trifluoromethoxy, arylalkyl, and alkylaryl. Preferably an aryl group consists of 6 to 12 carbon atoms.

For purposes of this invention the term “heteroaryl” is defined as an aromatic heterocyclic ring system (monocyclic or bicyclic) where the heteroaryl moieties are five or six membered rings containing 1 to 4 heteroatoms selected from the group consisting of S, N, and O, and include but is not limited to: (1) furan, thiophene, indole, azaindole, oxazole, thiazole, isoxazole, isothiazole, imidazole, N-methylimidazole, pyridine, pyrimidine, pyrazine, pyrrole, N-methypyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, pyrrolidinyl; (2) a bicyclic aromatic heterocycle where a phenyl, pyiidine, pyrimidine or pyridizine ling is: (i) fused to a 6-membered aromatic (unsaturated) heterocyclic ring having one nitrogen atom; (ii) fused to a 5 or 6-membered aromatic (unsaturated) heterocyclic ring having two nitrogen atoms; (iii) fused to a 5-membered aromatic (unsaturated) heterocyclic ring having one nitrogen atom together with either one oxygen or one sulfur atom; or (iv) fused to a 5-membered aromatic (unsaturated) heterocyclic ling having one heteroatom selected from O, N or S. Preferred substituents for heteroaiyl include: allcyl, halogen, amino, alkylamino, diallcylamino, trifluoromethyl, trifluoromethoxy, arylalkyl, and alcylaryl. A preferred heteroaryl moiety contains 1 to 9 carbon atoms.

For purposes of this invention the term “heterocycloalkyl” refers to a substituted or unsubstituted alicyclic ring system (moncyclic or bicyclic) wherein the heterocycloalkyl moieties are 3 to 12 membered lings containing 1 to 6 heteroatoms selected fiom the group consisting of S, N, and O. A preferred heterocycloalkyl moiety contains 1 to 9 carbon atoms, and more preferably contains 2 to 5 carbon atoms. Examples include, but are not limited to, pyrroline, pyrrolidine, imidazoline, imidazolidine, pyrazoline, pyrazolidine, pyran, dioxane, morpholine, dithiane, and thiomorpholine.

For the purposes of this invention the term “alkoxy” is defined as C₁-C₁₂-alkyl-O—, but preferably consists of 1 to 8 carbon atoms; the term “aryloxy” is defined as aryl-O—; the term “heteroaryloxy” is defined as heteroaryl-O—; the term “cycloalkoxy” is defined as cycloalkyl-O—; the term “heterocycloalkoxy” is defined as heterocycloalkyl-O—; wherein alkyl, aryl, cycloalkyl, heterocycloalkyl and heteroaryl are as defined above.

For the purpose of this invention the term “haloalkyl” refers to an alkyl moiety substituted with one or more halogenoatoms. An example of haloalkyl moiety is trifluoromethyl. The term “haloalkoxy” refers to an alkoxy moiety substituted with one or more halogen atoms, such as trifluoromethoxy.

The term “substituent” is used herein to refer to an atom radical, a functional group radical or a moiety radical that replaces a hydrogen radical on a molecule. Unless expressly stated otherwise, it should be assumed that any of the substituents may be optionally substituted with one or more groups selected from: alkyl, halo, nitro, amino, hydroxyl, cyano, alkylamino, dialkylamino, alkoxy, haloalkoxy, alkylthio, mercapto, haloalkylthio, aryl, aryloxy, arylthio, heteroaryl, heteroaryloxy, heteroarylthio or acyl. This list is provided for illustrative purposes and is not intended to be exhaustive.

For the purposes of this invention the term “substituted” refers to where a hydrogen radical on a molecule has been replaced by another atom radical, a functional group radical or a moiety radical; these radicals being generally referred to as “substituents.”

For the purposes of this invention the phrase “crude yield” refers to the percentage of starting material converted to the final product as calculated prior to purification by recrystalization.

Salts may be formed fiom addition of organic and inorganic acids. For example salts can be formed from the addition of acids, including but not limited to, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids. The most preferable acids for forming salts are acetic acid and hydrochloric acid.

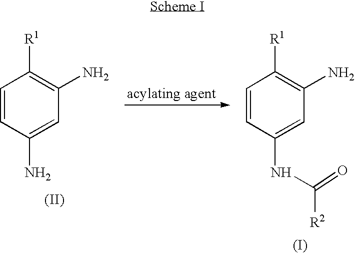

Scheme I illustrates the selective acylation of the 1-amino group, wherein R¹ and R² are as defined herein, of a 1,3-diamino phenyl compound of formula (II). The 1,3-diamino compound is reacted with an acylating agent, such as, for example, acetic anhydride, acetyl chloride, benzoyl chloride, ethyl chloroformate and pivaloyl chloride. Prefereably this reaction is conducted in the presence of a base. One skilled in the art would know of appropriate bases for use in this reaction, however, a tertiary amine base is preferable, such as triethylamine and pyridine. Pyridine is the most preferred.

In a preferred embodiment of the method of the present invention R¹ is NO₂.

This reaction can be conducted in an aprotic organic solvent. Commonly used solvents include methylene chloride, chloroform, CH₃CN, diethyl ether, THF, and tolene, or combinations thereof. This is not an all inclusive list and one skilled in the art would know of other useable solvents.

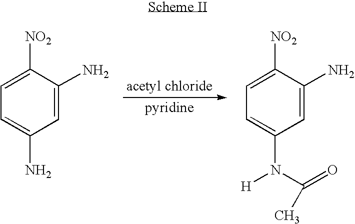

Scheme II shows the specific conversion of 4-nitro-1,3-phenylenediamine to 2-amino-4-acetimidonitrobenzene by adding acetyl chloride to a cooled solution of the starting material, 17% CH₃CN/THF and pyridine. This reaction was complete by the time the last of the acid chloride had been added. The reaction was then quenched with water, forcing the product to precipitate. The precipitate was collected by filtration and recrystallized using acetic acid to give product in a 69% yield. Other solvents can be used for the recrystallization, such as 23% v/v aqueous methoxyethanol and toluene. This is not an all inclusive list and one skilled in the art would know of other possible recrystallizing solvents or solutions.

### EXAMPLE 1

Preparation of 2-amino-4-acylimidonitrobenzene using an acid chloride.

A THF solution of the acid chloride (1.2 equivalents, 2.4 M) was added to an ice/water-cooled solution of 4-nitro-1,3-phenylenediamine (0.3 M) and pyridine (4.0 equivalents) in 17% v/v CH₃CN/THF. The reaction mixture was added to water after the starting material was consumed (as measured by TLC), causing the product to precipitate. The product was collected by filtration. The product formed using acetyl chloride was recrystallized from acetic acid (8 mL/g), when benzoyl chloride was used the product was recrystalized fiom 23% v/v aqueous methoxyethanol (13 mL/g), and the products formed from ethyl chlororformate and pivaloyl were recrystallized from toluene (14 mL/g). Yields are shown in Table 1.

### EXAMPLE 2

Preparation of 2-amino-4-acetimidonitrobenzene using acetic anhydride.

This reaction was performed as in Example 1 except acetic anhydride was used in place of an acid chloride. Under these conditions the reaction required 2 days to be completed. The product was recrystallized using acetic acid (8 mL/g). Yields are shown in Table 1.

### EXAMPLE 3

Preparation of 2-amino-4-trifluoroacetimidonitrobenzene using trifluoroacetic anhydride.

This reaction was performed as in Example 1 except trifluoroacetic anhydride (1.0 equiv.) was used in place of an acid chloride and the reaction was performed at −78° C. Under these conditions the reaction resulted in a 1:1:1 mixture of the mono-acylated products and the 1,3-bistrifluoroacetamide product (as measured by GC/MS).

TABLE 1
Yields and Purities

| Compound | acylating | | Yield, % | | Purity, % | |
|---|---|---|---|---|---|---|
| No. | agent | R² | ^{a} | ^{b} | ^{c} | mp (° C.) |
| 1 | acetic | —COMe | 88 | — | 97.9 | 206-207^{d} |
| | anhydride | | | | | |
| 1 | acetyl | —COMe | 69 | 69 | — | — |
| | chloride | | | | | |
| 2 | benzoyl | —COPh | 100 | 89 | 98.3 | 211-212 |
| | chloride | | | | | |
| 3 | ethyl | —CO₂Et | 96 | 79 | 98.5 | 182-183 |
| | chloroformate | | | | | |
| 4 | pivaloyl | —COC(Me)₃ | 90 | 81 | 98.5 | 166-167 |
| | chloride | | | | | |
| 5 | trifluoroacetic | —COCF₃ | ^{e} | — | — | — |
| | anhydride | | | | | |

^{a} Crude.
^{b} Recrystallized.
^{c} Determined by HPLC.
^{e} The products were not isolated.
GC/MS indicated that the crude reaction mixture contained a 1/1/1 mixture of starting material and the mono-and di-acylated compound.

Table 1 shows the results from reactions of 4-nitro-1,3-phenylenediamine with various acylating agents.

¹H and ¹³C NMR data for the compounds by the reaction of 4-nitro-1,3-phenylenediamine with various acylating agents:

Compound No. 1 ¹H NMR (300 MHz, d₆- DMSO) δ 2.70 (s, 3H), 6.65 (d,J=9.0 Hz, 1H), 7.48 (s, 2H), 7.54 (s, 1H), 7.91 (d,J=9.0 Hz, 1H), 10.17 (s, 1H); ¹³C NMR(75.5 MHz, d₆- DMSO) δ 170.0, 148.4, 146.1, 127.3, 126.7, 108.9, 106.1, 24.93.

Compound No. 2 ¹H NMR (300 MHz, d₆- DMSO) δ 6.94 (δ,J=9.0 Hz, 1H), 7.54-7.64 (m, 5H), 7.78 (s, 1H), 7.94-7.99 (m, 3H), 10.47 (s, 1H); ¹³CNMR (75.5 MHz, d₆- DMSO) δ 167.0, 148.2, 146.2, 135.2, 132.7, 129.1, 128.6, 127.1, 127.1, 110.0, 107.5.

Compound No. 3 ¹H NMR (300 MHz, d₆- DMSO) δ 1:25 (t,J=7.0 Hz, 3H), 4.15 (q,J=7.0 Hz, 2H), 6.62 (dd,J=7.8 and 1.5 Hz), 7.32 (d,J=1.5 Hz), 7.48 (s, 2H), 7.89 (d,J=7.8 Hz), 10.0 (s, 1H); ¹³C NMR (75.5 MHz, d₆- DMSO) δ 153.9, 148.4, 146.5, 127.4, 126.5, 108.4, 104.8, 61.4, 15.0.

Compound No. 4 ¹H NMR (300 MHz, d₆- DMSO) δ 1.22 (s, 9H), 6.82 (dd, J=7.6 and 1.8 Hz, 1H), 7.45 (s, 1H), 7.63 (d,J=1.8 Hz, 1H), 7.91 (d,J=7.6 Hz, 1H), 9.40 (s, 1H), ¹³C NMR (75.5 MHz, d₆- DMSO) δ 177.9, 148.2, 146.4, 126.9, 126.8, 109.9, 107.2, 27.6.

The examples disclosed in this application are for illustrative purposes so that the subject matter may be more readily understood and should not be construed to limit the scope of the invention as claimed herein.

What is claimed is:

1. A method of selectively acylating a compound of formula (II): 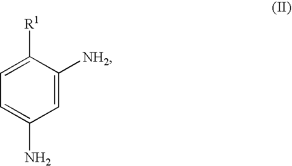
wherein:
R¹ is NO₂, —N⁺R³₃, trihalomethyl, —CN, —SO₃H, —CO₂H, —CO₂ R³, —CHO and —COR³, wherein R³ is C₁-C₆ alkyl, C₁-C₆ haloalkyl, C₃-C₁₂ cycloalkyl, C₆-C₁₂ aryl, C2-C₉ heteroaryl, or C₁-C₉ heterocycloalkyl;
complising the step of reacting the compound of formula (II) with an acylating reagent to form a compound of formula (I): 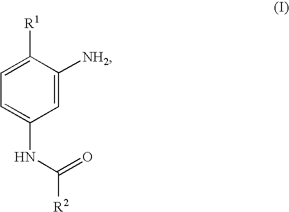
wherein R² is selected from C₁-C₁₂ alkyl, C₁-C₁₂ haloalkyl, C₂-C₇ alkenyl, C₂-C₇ alkynyl, C₃-C₁₂ cycloalkyl, C₆-C₁₂ aryl, C₁-C₉ heterocycloalkyl, C₂-C₉ heteroaryl, C₁-C₁₂ alkoxy, C₁-C₁₂ haloalkoxy, C₃-C₁₂ cycloalkoxy, C₁-C₉ heterocycloalkoxy, C₆-C₁₂ aryloxy, and C₂-C₉ heteroaryloxy;
or salts thereof.

2. The method of claim 1, further comprising that the acylation occurs in the presence of a base.

3. The method of claim 2, wherein the base is a tertiary amine.

4. The method of claim 3, wherein the base is pyridine.

5. The method of claim 1, wherein the acylating reagent is selected from the group consisting of acetic anhydride, acetyl chloride, beizoylchloride, ethylchloroformate, and pivaloyl chloride.

6. The method of claim 5, wherein the acylating reagent is acetyl chloride.

7. The method of claim 1, wherein the crude yield of the compound of formula (I) is at least about 60%.

8. The method of claim 1, wherein the crude yield of the compound of foimula (I) is at least about 70%.

9. The method of claim 8, wherein the crude yield of the compound of fonnula (I) is at least about 80%.

10. The method of claim 9, wherein the crude yield of the compound of formula (I) is at least about 90%.

11. The method of claim 1, further comprising the step of recrystallizing the compound of formula (I).

12. The method of claim 1, wherein the compound of formula (I) is recrystallized in a solvent selected from the group consisting of acetic acid, an aqueous methoxyethanol solution and toluene.

13. The method of claim 1, wherein R¹ is NO₂.
